(19) United States
(12) Patent Application Publication
(10) Pub. No.: US 2025/0136031 A1
(43) Pub. Date: May 1, 2025

(54) OCCUPANT MONITORING SYSTEM FOR A VEHICLE

(71) Applicant: GM Global Technology Operations LLC, Detroit, MI (US)

(72) Inventors: Akilesh Rajavenkatanarayanan, Macomb, MI (US); Joseph F. Szczerba, Grand Blanc, MI (US); Manoj Kumar Sharma, Troy, MI (US); Maureen Elizabeth August, Gross Pointe Woods, MI (US)

(21) Appl. No.: 18/497,417

(22) Filed: Oct. 30, 2023

(51) Int. Cl. B60R 21/015; A61B 5/00; A61B 5/0205; A61B 5/18; B60W 50/14; B60W 60/00; G06V 20/59; G06V 40/16

(52) U.S. Cl. CPC B60R 21/01538; A61B 5/0205; A61B 5/6893; A61B 5/7282; A61B 5/746; B60W 50/14; B60W 60/0051; G06V 20/59; G06V 40/172; A61B 5/18; B60R 2300/20; B60W 2420/403; B60W 2540/221

(57) ABSTRACT

An occupant monitoring system for monitoring one or more occupants located in a seat of a vehicle includes one or more cameras positioned to capture image data representative of an occupant, one or more biometric sensors that each monitor a parameter indicative of a physiological measure of the occupant, and one or more controllers. The one or more controllers execute instructions to determine one or more physiological measures of the occupant based on the image data collected by the one or more cameras and one or more parameters indicative of the one or more physiological measures of each occupant collected by the one or more biometric sensors, and in response to determining the physiological measure exceeds either the major event threshold value or the minor event threshold value, generate a notification directed to a driver of the vehicle.

### INTRODUCTION

The present disclosure relates to an occupant monitoring system that monitors one or more physiological measures of one or more occupants located in a seat of a vehicle.

Children who are twelve years old and younger, or who are below a minimum height, should typically be placed in one of the rear seats of a vehicle. In many situations, the caregiver of the child placed in the rear seat is operating the vehicle as a driver. As a result, the caregiver may have to turn his or her head away from the road viewed through the windscreen to view the child, who is seated in the back of the vehicle. Accordingly, it may be challenging for the caregiver to pay attention or attend to the child while still operating the vehicle. For example, a very young child, such as an infant or toddler, may become upset, agitated, or throw a tantrum while riding in a vehicle. It may be difficult for the caregiver to pacify the young child while still operating the vehicle. Furthermore, it is to be appreciated that very young children and other individuals, such as the elderly, may not be able to verbally communicate their health issues or when they are experiencing discomfort. This may also create issues since the caregiver is unable to check on or attend to the individual while operating the vehicle.

Thus, while current vehicles achieve their intended purpose, there is a need in the art for an improved approach to monitor rear seat occupants by a driver of a vehicle.

### SUMMARY

According to several aspects, an occupant monitoring system for monitoring one or more occupants located in a seat of a vehicle is disclosed. The occupant monitoring system includes one or more cameras positioned to capture image data representative of an occupant, one or more biometric sensors that each monitor a parameter indicative of a physiological measure of the occupant, and one or more controllers in electronic communication with the one or more cameras and the one or more biometric sensors. The one or more controllers execute instructions to determine one or more physiological measures of an occupant based on the image data collected by the one or more cameras and one or more parameters indicative of the one or more physiological measures of each occupant collected by the one or more biometric sensors. The one or more controllers compare a physiological measure of the occupant with a baseline value, where the baseline value for each physiological measurement is indicative of the occupant functioning normally. In response to determining the physiological measure falls outside an allowable range of the baseline value, the one or more controllers compare the physiological measure with at least one of a major event threshold value and a minor event threshold value. In response to determining the physiological measure exceeds either the major event threshold value or the minor event threshold value, the one or more controllers generate a notification directed to a driver of the vehicle.

In another aspect, the one or more controllers execute instructions to determine the physiological measure exceeds the major event threshold value, and in response to determining the physiological measure exceeds the major event threshold value, determine the occupant requires immediate action.

In yet another aspect, the notification includes a visual alert and an audio alert.

In an aspect, the one or more controllers execute instructions to instruct the vehicle to perform one or more of the following corrective actions: switch into a fully autonomous mode of driving so the driver may attend to the occupant, navigate the vehicle to the nearest hospital, contact emergency personnel by an in-vehicle wireless communication system, and perform an automated pull-over to the side of the road by an automated driving system.

In another aspect, the one or more controllers execute instructions to determine the physiological measure exceeds the minor event threshold value, and in response to determining the physiological measure exceeds the minor event threshold value, determine the occupant is experiencing a physical issue that requires monitoring by the driver of the vehicle.

In another aspect, the notification includes includes a visual message informing the driver of the physiological measure has exceeded the minor event threshold value.

In yet another aspect, the occupant monitoring system further includes a front row display in electronic communication with the one or more controllers, wherein the front row display generates visual content visible by a driver of the vehicle.

In an aspect, the front row display is one of the following: an augmented reality head-up display (AR-HUD) and an infotainment screen.

In another aspect, the one or more controllers execute instructions to instruct the front row display to generate visual content representative of the physiological measures of each of the one or more occupants.

In yet another aspect, the visual content includes a live video feed that is based on the image data captured by the one or more cameras.

In an aspect, the occupant monitoring system further comprises one or more occupant databases in electronic communication with the one or more controllers storing facial recognition data representative of facial features of one or more known individuals and data indicating baseline values for the physiological measures that correspond to a specific known individual.

In another aspect, the one or more controllers execute instructions to execute one or more facial recognition algorithms to determine an identity of the occupant based on the image data captured by the one or more cameras and compare the identity of the occupant with the facial recognition data of the one or more known individuals stored in the one or more occupant databases.

In yet another aspect, in response to determining a match exists between the identity of the occupant and the facial recognition data representative of one of the known individuals, the one or more controllers utilize the baseline values for the physiological measures corresponding to the specific known individual stored in the one or more occupant databases.

In an aspect, in response to determining no match exists, the one or more controllers utilize default baseline values for the physiological measures.

In another aspect, the one or more cameras include one or more of the following: a red, green, and blue (RGB) camera that captures visible light image data, an infrared camera that captures infrared image data, and a thermal camera that captures thermal image data.

In yet another aspect, the physiological measures include one or more of the following: heart rate, respiratory rate, body temperature, muscle stiffness, sweat rate, drowsiness, stress, motion sickness, pupil dilation, blood sugar levels, and blood pressure.

In an aspect, the occupant monitoring system further comprises one or more radar sensors in electronic communication with the one or more controllers, wherein the one or more radar sensors generate signals indicative of the presence and a seat position of the occupant.

In another aspect, a method of monitoring one or more occupants located in a seat of a vehicle by an occupant monitoring system is disclosed. The method includes determining, by one or more controllers, one or more physiological measures of an occupant based on image data collected by one or more cameras and one or more parameters indicative of one or more physiological measures of each occupant collected by one or more biometric sensors. The method includes comparing, by the one or more controllers, a physiological measure of the occupant with a baseline value, where the baseline value for each physiological measurement is indicative of the occupant functioning normally. In response to determining the physiological measure falls outside an allowable range of the baseline value, the method includes comparing the physiological measure with a major event threshold value and a minor event threshold value. In response to determining the physiological measure exceeds either the major event threshold value or the minor event threshold value, the method includes generating a notification directed to a driver of the vehicle.

In another aspect, an occupant monitoring system for monitoring one or more occupants located in a seat of a vehicle is disclosed. The occupant monitoring system includes one or more cameras positioned to capture image data representative of an occupant, one or more biometric sensors that each monitor a parameter indicative of a physiological measure of the one or more occupant, and one or more controllers in electronic communication with the one or more cameras and the one or more biometric sensors. The one or more controllers execute instructions to determine one or more physiological measures of an occupant based on the image data collected by the one or more cameras and one or more parameters indicative of the one or more physiological measures of each occupant collected by the one or more biometric sensors. The one or more controllers compare a physiological measure of the occupant with a baseline value, wherein the baseline value for each physiological measurement is indicative of the occupant functioning normally. In response to determining the physiological measure falls outside an allowable range of the baseline value, the one or more controllers compare the physiological measure with a major event threshold value. In response to determining the physiological measure exceeds the major event threshold value, the one or more controllers generate a notification directed to a driver of the vehicle and determine the occupant requires immediate action.

In another aspect, in response to determining the physiological measure does not exceed the major event threshold value, the one or more controllers compare the physiological measure to a minor event threshold value, determine the physiological measure exceeds the minor event threshold value, and in response to determining the physiological measure exceeds the minor event threshold value, determine the occupant is experiencing a physical issue that requires monitoring by the driver of the vehicle.

Further areas of applicability will become apparent from the description provided herein. It should be understood that the description and specific examples are intended for purposes of illustration only and are not intended to limit the scope of the present disclosure.

### BRIEF DESCRIPTION OF THE DRAWINGS

The drawings described herein are for illustration purposes only and are not intended to limit the scope of the present disclosure in any way.

FIG. 1 is a schematic diagram of a vehicle including the disclosed occupant monitoring system for monitoring one or more physiological measures of one or more occupants, according to an exemplary embodiment;

FIG. 2A is an exemplary illustration of an infotainment screen of the occupant monitoring system, where the infotainment screen displays visual content representative of the physiological measures of each occupant located in the vehicle, according to an exemplary embodiment;

FIG. 2B is an exemplary illustration of another embodiment of the infotainment screen shown in FIG. 2A where the infotainment screen displays the physiological measures of each occupant based on a seat position, according to an exemplary embodiment;

FIG. 2C is an exemplary illustration of another embodiment of the infotainment screen shown in FIG. 2A where the infotainment screen displays a live video feed of image data representative of the one or more occupants, according to an exemplary embodiment; and

FIG. 3 illustrates a process flow diagram illustrating a method for monitoring one or more occupants located in the vehicle shown in FIG. 1 by the disclosed occupant monitoring system, according to an exemplary embodiment.

### DETAILED DESCRIPTION

The following description is merely exemplary in nature and is not intended to limit the present disclosure, application, or uses.

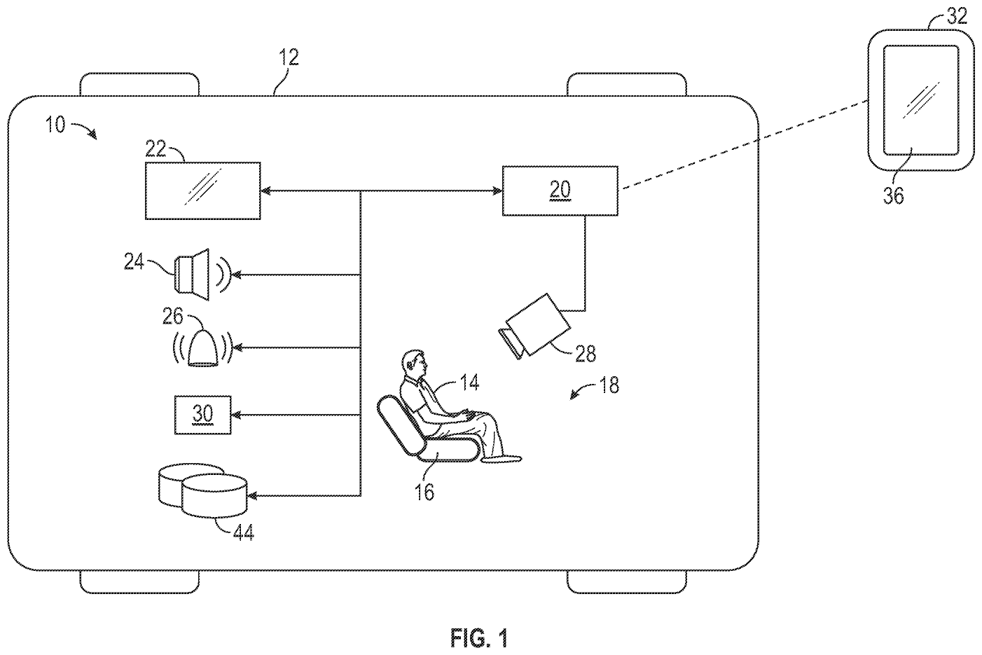

Referring to FIG. 1, an occupant monitoring system 10 for a vehicle 12 is illustrated. As explained below, the occupant monitoring system 10 monitors one or more physiological measures of one or more occupants 14 located in one of the seats 16 of the vehicle 12. In one non-limiting embodiment, the seat 16 is part of second row seating, third row seating, or fourth row seating in the vehicle 12. In an embodiment, the one or more occupants 14 being monitored are located within an interior cabin 18 of the vehicle 12 but are not seated upon any of the seats in the interior cabin 18. In one embodiment, the one or more occupants 14 include an individual who requires close attention such as, for example, an infant, a toddler, a young child under 12 years of age, or a very elderly individual. In the alternative, the one or more occupants 14 may include an animal such as a pet like a dog or a cat. It is to be appreciated that the vehicle 12 may be any type of vehicle such as, but not limited to, a sedan, a truck, sport utility vehicle, van, bus, or motor home.

The occupant monitoring system 10 includes one or more controllers 20 in electronic communication with a front row display 22, a speaker 24, one or more radar sensors 26, one or more cameras 28, and one or more biometric sensors 30. In one embodiment, the one or more controllers 20 are in wireless communication with a personal electronic device 32 associated with the driver of the vehicle 12. The personal electronic device 32 is any type of portable electronic device having wireless capabilities and including one or more processors and a display 36 that shows images and graphics to the driver such as, for example, a smartphone or a tablet computer.

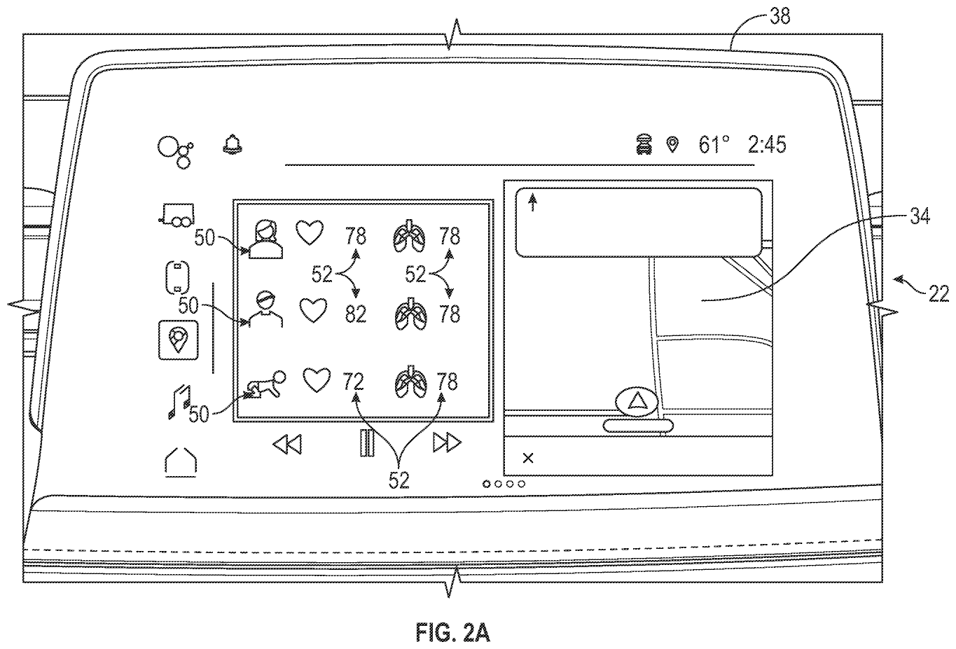

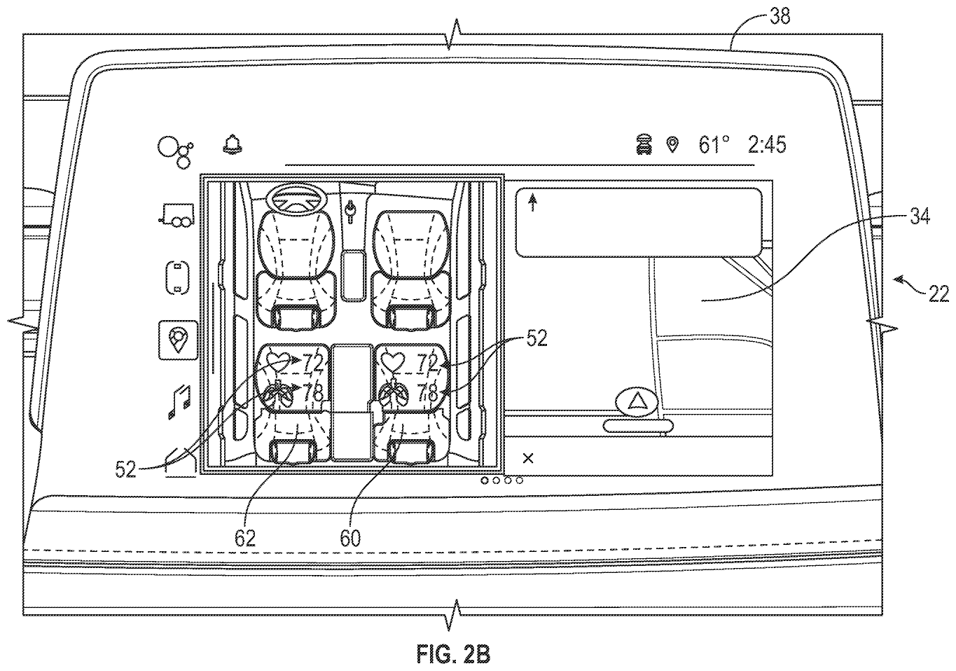

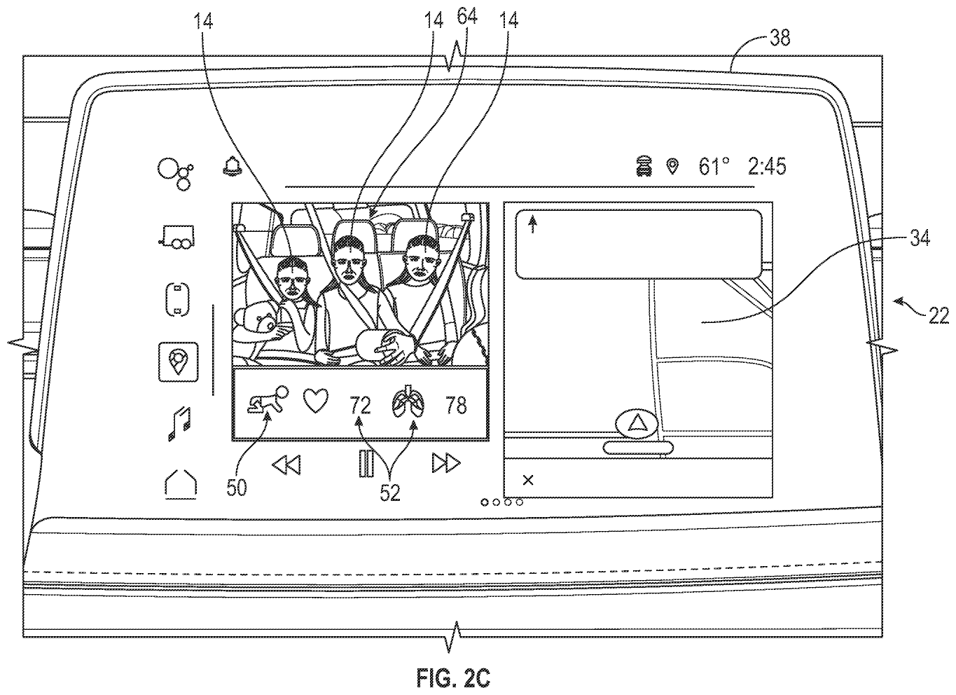

The front row display 22 is positioned within the interior cabin 18 of the vehicle 12 and generates visual content such as graphics and text that are visible by the driver of the vehicle 12. It is to be appreciated that the visual content generated by the front row display 22 is visible as the driver operates the vehicle 12 without the need for the driver to turn his or her head or body towards the rear of the vehicle 12. In one non-limiting embodiment, the front row display 22 is an augmented reality head-up display (AR-HUD) that generates the visual content directly onto a windscreen of the vehicle 12 within the driver's forward field of view. Alternatively, in another embodiment, the front row display 22 includes an infotainment screen 34 (FIG. 2A) that shows the visual content, where the infotainment screen is mounted to a dashboard 38 of the vehicle 12.

The one or more radar sensors 26 provide signals indicative of the presence and a seat position of the occupant 14 to the one or more controllers 20. The one or more cameras 28 are located within the interior cabin 18 of the vehicle 12 and are positioned to capture image data representative of the occupant 14 located in the seats 16. The one or more cameras 28 include red, green, and blue (RGB) cameras that capture visible light image data, infrared cameras that capture infrared image data, or thermal cameras that capture thermal image data. It is to be appreciated that the image data includes visible light image data, infrared image data, and thermal image data.

The one or more biometric sensors 30 each monitor a parameter indicative of a physiological measure of the occupant 14 located in the seat 16 of the vehicle 12. The physiological measure represents a bodily function of an individual. Some examples of physiological measures of the occupant 14 include, but are not limited to, heart rate, respiratory rate, body temperature, muscle stiffness, sweat rate, drowsiness, stress, motion sickness, pupil dilation, blood sugar levels, and blood pressure. It is to be appreciated that in embodiments, the one or more biometric sensors 30 include the one or more radar sensors 26 and the one or more cameras 28. Some examples of the one or more biometric sensors 30 include, but are not limited to, a heart rate sensor, a galvanic skin response (GSR) sensor that detects an electrical conductance of the skin of the occupant 14, and a piezoelectric muscle sensor that detects a piezoelectric resonance of the occupant 14, where the piezoelectric resonance is indicative of muscle stiffness. In embodiments, the GSR sensor is mounted to a surface of the door handles or an armrest to contact the skin of an occupant 14.

In embodiments, the one or more controllers 20 determine the sweat rate of the occupant 14 based on thermal image data representative of a face of the occupant 14, and specifically a forehead of the occupant 14, which is captured by the one or more thermal imaging cameras 28. In embodiments, the sweat rate of the occupant 14 is further determined based on visible light image data captured by the one or more RGB cameras 28 as well.

In embodiments, the drowsiness of the occupant 14 is determined based on the image data captured by the one or more cameras 28, the heart rate of the occupant detected by the heart rate sensor, and the electrical conductance of the skin of the occupant 14 measured by the GSR sensor. For example, the image data captured by the one or more cameras 28 is indicative of various behaviors of the occupant 14 that indicate drowsiness such as, but not limited to, yawning, eye closure, eye blinking, and head pose. Furthermore, it is also to be appreciated that the heart rate of the occupant 14 varies significantly between different stages of drowsiness, such as alertness and fatigue.

In one embodiment, the one or more controllers 20 determine the stress of the occupant 14 based on the heart rate of the occupant detected by the heart rate sensor, and the electrical conductance of the skin of the occupant 14 measured by the GSR sensor. Alternatively, in another embodiment, the one or more controllers 20 determine the stress of the occupant 14 based on the image data collected by the one or more cameras 28 as well. In an embodiment, motion sickness of the occupant 14 is determined based on pupillary rhythms that are monitored by the image data captured by the one or more cameras 28.

Continuing to refer to FIG. 1, during initialization of the occupant monitoring system 10, the one or more controllers 20 monitor the image data representative of the occupant 14 located in the seat 16 captured by the one or more cameras 28. The one or more controllers 20 execute one or more facial recognition algorithms to determine an identity of the occupant 14 based on the image data representative of the occupant 14. The one or more controllers 20 are in electronic communication with one or more occupant databases 44 that store facial recognition data representative of the facial features of one or more known individuals. The one or more occupant databases 44 also store data indicating baseline values for the physiological measures that correspond to a specific known individual. In embodiments, the occupant databases 44 are stored locally, however, the occupant databases 44 may be stored remotely on a network server as well.

The one or more controllers 20 execute instructions to compare the identity of the occupant 14 with the facial recognition data stored in the one or more occupant databases 44. In response to identifying a match between the identity of the occupant 14 and one of the known individuals stored in the occupant database 44, the one or more controllers 20 utilize the baseline values for the physiological measures corresponding to the specific known individual stored in the one or more occupant databases 44. However, if no match is identified, then the one or more controllers 20 utilize default baseline values for the physiological measurements for the occupant 14.

The one or more controllers 20 execute instructions to determine one or more physiological measures of each occupant 14 based on one or more of the following: the signals received from the one or more radar sensors 26, the image data collected by the one or more cameras 28, and the one or more parameters indicative of the one or more physiological measures of each occupant 14 collected by the one or more biometric sensors 30. The one or more controllers 20 instruct the front row display 22 to generate visual content representative of the physiological measures of each occupant 14. For example, FIG. 2A is an exemplary illustration of the front row display 22, where the front row display 22 includes the infotainment screen 34 displaying the visual content representative of the physiological measures of each occupant 14 located in the vehicle 12. In the example as shown in FIG. 2A, the infotainment screen 34 displays an icon 50 corresponding to one of the occupants 14 and one or more icons 52 representing physiological measures corresponding to each occupant 14 located within the interior cabin 18 of the vehicle 12.

In the example as shown in FIG. 2A, the icon 50 represents a gender of the corresponding occupant 14 and the one or more icons 52 represent a heart rate and a respiratory rate corresponding to each occupant 14 in the vehicle 12. However, it is to be appreciated that the driver or another occupant may customize the front row display 22 so the icons 52 represent different or additional physiological measures instead.

FIG. 2B is an alternative embodiment of the infotainment screen 34 shown in FIG. 2A, where the infotainment screen 34 displays the physiological measures of each of the occupants 14 based on seat position. In the example as shown in FIG. 2B, a rear passenger side seat position 60 and a rear driver side seat position 62 are shown, where each seat position 60, 62 is associated with one or more icons 52 representing physiological measures corresponding to a respective occupant 14.

FIG. 2C is yet another embodiment of the infotainment screen 34 shown in FIG. 2A, where the infotainment screen 34 displays a live video feed 64 based on the image data captured by the one or more cameras 28 (FIG. 1) of the occupants 14. The infotainment screen 34 also displays the icon 50 corresponding to a selected one of the occupants 14 and one or more icons 52 representing physiological measures for the selected occupant 14. It is to be appreciated that the driver may switch the view of the infotainment screen 34 to display the physiological measure of one of the remaining occupants 14, if applicable, using a touchscreen feature, button, or based on an automatic toggle feature.

Referring to FIG. 1, the one or more controllers 20 execute instructions to continuously compare each physiological measure of the occupant 14 with a baseline value. The baseline value corresponds to a specific physiological measurement and is indicative of the occupant 14 functioning normally and is not experiencing a health-related event. That is, in other words, the baseline value corresponding to a specific physiological measurement indicates occupant 14 does not have a physiological issue that requires immediate action or requires monitoring by the driver. Some examples of physiological issues that require monitoring by the driver include an elevated heart rate, elevated body temperature, and high stress. It is to be appreciated that physiological issues that require monitoring by the driver do not require actual intervention. In contrast, sometimes the occupant 14 may undergo a physiological issue that requires the driver to intervene, such as a heart attack, anxiety attack, or an asthma attack. In embodiments, the baseline value indicates an allowable range of values for a corresponding physiological measure that indicates an individual is functioning normally.

In response to determining a physiological measure falls outside the allowable range of the baseline values, the one or more controllers 20 compare the physiological measure with a major event threshold value. The one or more controllers 20 also compare the physiological measure with a minor event threshold value. The major event threshold value is indicative of the occupant 14 experiencing a physiological issue that requires immediate action from the driver of the vehicle 12. In response to determining the physiological measure exceeds the major event threshold value, the one or more controllers 20 determine the occupant 14 requires immediate action. Some examples of physiological measures represented by the major event threshold value include, but are not limited to, a high-grade fever (over about 39.5° C. or 103° F.), or a relatively sharp drop in blood pressure (at least 20 millimeters of mercury (mmHg)).

In response to determining the occupant 14 requires immediate action, the one or more controllers 20 generate a notification directed to the driver of the vehicle 12. In one embodiment, the notification includes a visual alert created by the front row display 22 and an audio alert generated by the speaker 24. For example, the visual alert may include an emergency graphic and a message stating the occupant 14 requires immediate action, and the audio alert may include an alarm, tone, or voice message that indicates immediate action is required. In one embodiment, the one or more controllers 20 may transmit a signal instructing the personal electronic device 32 associated with the driver of the vehicle 12 to also generate the visual alert, the audio alert, or both. It is to be appreciated that the personal electronic device 32 may be used in situations when the driver has momentarily stepped away from the vehicle 12.

In embodiments, in response to determining the occupant 14 requires immediate action, the one or more controllers 20 instruct the vehicle 12 to perform one or more of the following corrective actions: switch into a fully autonomous mode of driving so the driver may attend to the occupant 14, navigate the vehicle 12 to the nearest hospital or emergency room, contact emergency personnel by an in-vehicle wireless communication system, and perform an automated pull-over to the side of the road by an automated driving system. In one embodiment, the one or more controllers 20 may instruct the front row display 22 to generate a graphic asking the driver of the vehicle 12 to select one of the corrective actions.

The minor event threshold value indicates the occupant 14 is experiencing a physiological issue that requires monitoring by the driver of the vehicle 12 but does not require immediate action. In response to determining the physiological measure exceeds the minor event threshold value, the one or more controllers 20 generate a notification. When the occupant 14 does not require immediate attention, in one embodiment the notification includes a visual message generated by the front row display 22 informing the driver of the physiological measure of the occupant 14. As seen in FIG. 20, in an embodiment the front row display 22 generates an icon 50 corresponding to the occupant 14 and an icon 52 representing the physiological measures. In an embodiment, the front row display 22 also shows the live video feed 64 of the image data captured by the one or more cameras 28 (FIG. 1) representative of the occupant 14. Some examples of physiological measures represented by the minor event threshold value include, but are not limited to, a low-grade fever (ranging between 37.7-39.5° C. or 100-103° F.).

In one embodiment, the one or more controllers 20 execute one or more object detection and tracking algorithms to identify a relevant object within the control of the occupant 14, track the relevant object, and determine when the occupant 14 has lost control of the relevant object based on the image data captured by the one or more cameras 28. For example, in one embodiment, the occupant 14 is a toddler, and the relevant object is a favorite toy or a pacifier. The one or more controllers 20 analyze the image data captured by the one or more cameras 28 to identify the relevant object, which is located within one or more of the hands or the lap of the occupant 14 based on one or more object detection algorithms. The one or more controllers 20 execute tracking algorithms to continuously track the relevant object. The one or more controllers 20 determine the occupant 14 has lost control of the relevant object in response to detecting the relevant object has dropped onto the floor of the interior cabin 18 of the vehicle 12. The one or more controllers 20 may also accomplish vomit detection, rear seat belt status monitoring, and determining if the occupant 14 is placing a body part through a window of the vehicle 12 based on one or more object detection and tracking algorithms as well.

In another embodiment, the one or more controllers 20 monitor the signals received from the one or more radar sensors 26, the image data collected by the one or more cameras 28, and the one or more parameters indicative of the one or more physiological measures of each occupant 14 collected by the one or more biometric sensors 30 to determine a comfort level of the occupant 14. The comfort level of the occupant 14 is determined based on the respiratory rate and the sweat rate of the occupant 14. It is to be appreciated that in some instances, the driver may momentarily step away from the vehicle 12, but the occupant 14 is still located in the seat 16. The one or more controllers 20 compare the comfort level with a threshold respiratory rate value and a threshold sweat rate value, where the threshold respiratory rate value and the threshold sweat rate value are indicative of occupant discomfort. In embodiments, in response to determining the comfort level exceeds either the threshold respiratory rate value or the threshold sweat rate value, the one or more controllers 20 instruct an in-cabin heating, ventilation, and air conditioning (HVAC) system to adjust an in-cabin temperature of the interior cabin 18. For example, in warmer temperatures the in-cabin HVAC increases cooling to the occupant 14 and in cooler temperatures the in-cabin HVAC increases heating to the occupant 14. In one embodiment, the one or more controllers 20 may transmit a signal instructing the personal electronic device 32 associated with the driver of the vehicle 12 to also generate the visual alert, the audio alert, or both indicating the occupant 14 is experiencing discomfort.

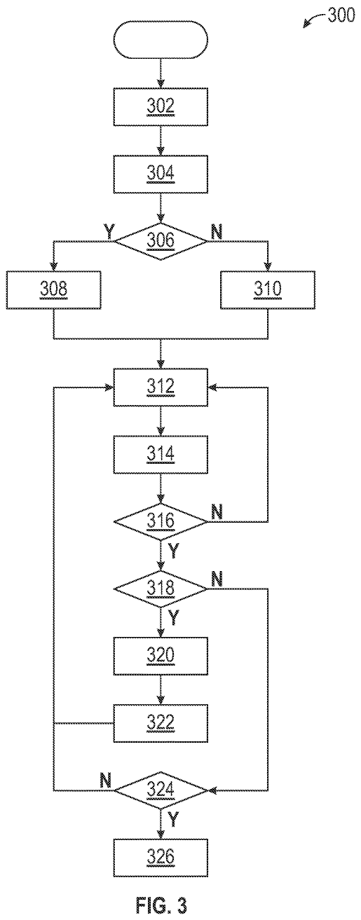

FIG. 3 is a process flow diagram illustrating a method 300 for monitoring the one or more occupants 14 located in the seat 16 of a vehicle 12 by the occupant monitoring system 10. Referring to both FIGS. 1 and 3, the method 300 may begin at block 302. In block 302, the one or more controllers 20 monitor the image data captured by the one or more cameras 28 representative of the occupant 14 located in the seat 16 of the vehicle 12. The method 300 may then proceed to block 302.

In block 302, the one or more controllers 20 execute one or more facial recognition algorithms to determine the identity of the occupant 14 based on the image data. The method 300 may then proceed to decision block 304.

In decision block 306, the one or more controllers 20 compare the identity of the occupant 14 with the facial recognition data of the one or more known individuals stored in the one or more occupant databases 44 to determine if a match exists between the identity of the occupant 14 and the facial recognition data representative of one of the known individuals. In response to determining a match exists, the method proceeds to block 308. In block 308, the one or more controllers 20 utilize the baseline values for the physiological measures corresponding to the specific known individual stored in the one or more occupant databases 44, and the method 300 proceeds to block 312.

In response to determining no match exists, the method proceeds to block 310. In block 310, the one or more controllers 20 utilize the default baseline values for the physiological measures. The method 300 may then proceed to block 312.

In block 312, the one or more controllers 20 execute instructions to determine one or more physiological measures of the occupant 14 based on one or more of the following: the signals received from the one or more radar sensors 26, the image data collected by the one or more cameras 28, and the one or more parameters indicative of the one or more physiological measures of each occupant 14 collected by the one or more biometric sensors 30. The method 300 may then proceed to block 314.

In block 314, the one or more controllers 20 instruct the front row display 22 to generate visual content representative of the physiological measures of each occupant 14, which is shown in FIGS. 2A-2C. The method 300 may then proceed to decision block 316.

In decision block 316, the one or more controllers 20 compare a physiological measure of the occupant 14 with a baseline value, where the baseline value for the physiological measurement is indicative of the occupant functioning normally. In response to determining the physiological measure falls outside the allowable range of the baseline value, the method 300 proceeds to decision block 318. Otherwise, the method 300 returns to block 312.

In decision block 318, the one or more controllers 20 compare the physiological measure with the major event threshold value. In response to determining the physiological measure exceeds the major event threshold value, the one or more controllers 20 determine the occupant 14 requires immediate action, and the method proceeds to block 320. Otherwise, the method 300 may proceed to decision block 324.

In block 320, in response to determining the occupant 14 requires immediate action, the one or more controllers 20 generate the notification directed to the driver of the vehicle 12. As mentioned above, in one embodiment the notification includes a visual alert created by the front row display 22 and an audio alert generated by the speaker 24. The method 300 may then proceed to block 322.

In block 322, the one or more controllers 20 instruct the vehicle 12 to perform one or more of the following corrective actions: switch into a fully autonomous mode of driving so the driver may attend to the occupant 14, navigate the vehicle 12 to the nearest hospital, contact emergency personnel by an in-vehicle wireless communication system, and perform an automated pull-over to the side of the road by an automated driving system. As mentioned above, in embodiments the one or more controllers 20 may instruct the front row display 22 to generate a graphic asking the driver of the vehicle 12 to select one of the corrective actions. The method 300 may then terminate or return to block 312.

In decision block 324, the one or more controllers 20 compare the physiological measure with the minor event threshold value. In response to determining the physiological measure exceeds the minor event threshold value, the one or more controllers 20 determine the occupant 14 is experiencing a physical issue that requires monitoring by the driver of the vehicle 12, and the method 300 proceeds to block 326. Otherwise, the method 300 returns to block 312.

In block 326, the one or more controllers 20 generate the notification directed to the driver of the vehicle 12. As mentioned above, the notification includes includes a visual message generated by the front row display 22 informing the driver of the physiological measure of the occupant 14 that has exceeded the minor event threshold value. As seen in FIG. 2C, in one embodiment the front row display 22 also shows the live video feed 64 of the image data captured by the one or more cameras 28 (FIG. 1) representative of the occupant 14. The method 300 may then terminate.

Referring generally to the figures, the disclosed occupant monitoring system provides various technical effects and benefits. Specifically, the occupant monitoring system provides an approach for the driver to monitor one or more occupants located in the vehicle, without the need for the driver to turn his or her head or body away from the windscreen of the vehicle. The occupant monitoring system also provides notifications to the driver when the occupant requires immediate action, or when the occupant is merely experiencing a physical issue that requires monitoring by the driver of the vehicle. It is to be appreciated that the occupant monitoring system may be especially advantageous when a very young child or an elderly person who is unable to verbally communicate is in the rear seat of the vehicle, since the occupant monitoring system requires no verbal communication or affirmative action to be completed by the rear occupant.

The controllers may refer to, or be part of an electronic circuit, a combinational logic circuit, a field programmable gate array (FPGA), a processor (shared, dedicated, or group) that executes code, or a combination of some or all of the above, such as in a system-on-chip. Additionally, the controllers may be microprocessor-based such as a computer having a at least one processor, memory (RAM and/or ROM), and associated input and output buses. The processor may operate under the control of an operating system that resides in memory. The operating system may manage computer resources so that computer program code embodied as one or more computer software applications, such as an application residing in memory, may have instructions executed by the processor. In an alternative embodiment, the processor may execute the application directly, in which case the operating system may be omitted.

The description of the present disclosure is merely exemplary in nature and variations that do not depart from the gist of the present disclosure are intended to be within the scope of the present disclosure. Such variations are not to be regarded as a departure from the spirit and scope of the present disclosure.

What is claimed is:

1. An occupant monitoring system for monitoring one or more occupants located in a seat of a vehicle, the occupant monitoring system comprising:
one or more cameras positioned to capture image data representative of an occupant;
one or more biometric sensors that each monitor a parameter indicative of a physiological measure of the occupant; and
one or more controllers in electronic communication with the one or more cameras and the one or more biometric sensors, wherein the one or more controllers execute instructions to:
determine one or more physiological measures of an occupant based on the image data collected by the one or more cameras and one or more parameters indicative of the one or more physiological measures of each occupant collected by the one or more biometric sensors;
compare a physiological measure of the occupant with a baseline value, wherein the baseline value for each physiological measurement is indicative of the occupant functioning normally;
in response to determining the physiological measure falls outside an allowable range of the baseline value, compare the physiological measure with at least one of a major event threshold value and a minor event threshold value; and
in response to determining the physiological measure exceeds either the major event threshold value or the minor event threshold value, generate a notification directed to a driver of the vehicle.

2. The occupant monitoring system of claim 1, wherein the one or more controllers execute instructions to:
determine the physiological measure exceeds the major event threshold value; and
in response to determining the physiological measure exceeds the major event threshold value, determine the occupant requires immediate action.

3. The occupant monitoring system of claim 2, wherein the notification includes a visual alert and an audio alert.

4. The occupant monitoring system of claim 2, wherein the one or more controllers execute instructions to:
instruct the vehicle to perform one or more of the following corrective actions: switch into a fully autonomous mode of driving so the driver may attend to the occupant, navigate the vehicle to the nearest hospital, contact emergency personnel by an in-vehicle wireless communication system, and perform an automated pull-over to the side of the road by an automated driving system.

5. The occupant monitoring system of claim 1, wherein the one or more controllers execute instructions to:
determine the physiological measure exceeds the minor event threshold value; and
in response to determining the physiological measure exceeds the minor event threshold value, determine the occupant is experiencing a physical issue that requires monitoring by the driver of the vehicle.

6. The occupant monitoring system of claim 5, wherein the notification includes a visual message informing the driver of the physiological measure has exceeded the minor event threshold value.

7. The occupant monitoring system of claim 1, further comprising a front row display in electronic communication with the one or more controllers, wherein the front row display generates visual content visible by a driver of the vehicle.

8. The occupant monitoring system of claim 7, wherein the front row display is one of the following: an augmented reality head-up display (AR-HUD) and an infotainment screen.

9. The occupant monitoring system of claim 7, wherein the one or more controllers execute instructions to:
instruct the front row display to generate visual content representative of the physiological measures of each of the one or more occupants.

10. The occupant monitoring system of claim 9, wherein the visual content includes a live video feed that is based on the image data captured by the one or more cameras.

11. The occupant monitoring system of claim 1, further comprising one or more occupant databases in electronic communication with the one or more controllers storing facial recognition data representative of facial features of one or more known individuals and data indicating baseline values for the physiological measures that correspond to a specific known individual.

12. The occupant monitoring system of claim 11, wherein the one or more controllers execute instructions to:
execute one or more facial recognition algorithms to determine an identity of the occupant based on the image data captured by the one or more cameras; and
compare the identity of the occupant with the facial recognition data of the one or more known individuals stored in the one or more occupant databases.

13. The occupant monitoring system of claim 12, wherein the one or more controllers execute instructions to:
in response to determining a match exists between the identity of the occupant and the facial recognition data representative of one of the known individuals, utilize the baseline values for the physiological measures corresponding to the specific known individual stored in the one or more occupant databases.

14. The occupant monitoring system of claim 12, wherein the one or more controllers execute instructions to:
in response to determining no match exists, utilize default baseline values for the physiological measures.

15. The occupant monitoring system of claim 1, wherein the one or more cameras include one or more of the following: a red, green, and blue (RGB) camera that captures visible light image data, an infrared camera that captures infrared image data, and a thermal camera that captures thermal image data.

16. The occupant monitoring system of claim 1, wherein the physiological measures include one or more of the following: heart rate, respiratory rate, body temperature, muscle stiffness, sweat rate, drowsiness, stress, motion sickness, pupil dilation, blood sugar levels, and blood pressure.

17. The occupant monitoring system of claim 1, further comprising one or more radar sensors in electronic communication with the one or more controllers, wherein the one or more radar sensors generate signals indicative of the presence and a seat position of the occupant.

18. A method of monitoring one or more occupants located in a seat of a vehicle by an occupant monitoring system, the method comprising:
determining, by one or more controllers, one or more physiological measures of an occupant based on image data collected by one or more cameras and one or more parameters indicative of one or more physiological measures of each occupant collected by one or more biometric sensors;
comparing, by the one or more controllers, a physiological measure of the occupant with a baseline value, wherein the baseline value for each physiological measurement is indicative of the occupant functioning normally;
in response to determining the physiological measure falls outside an allowable range of the baseline value, comparing the physiological measure with a major event threshold value and a minor event threshold value; and
in response to determining the physiological measure exceeds either the major event threshold value or the minor event threshold value, generating a notification directed to a driver of the vehicle.

19. An occupant monitoring system for monitoring one or more occupants located in a seat of a vehicle, the occupant monitoring system comprising:
one or more cameras positioned to capture image data representative of an occupant;
one or more biometric sensors that each monitor a parameter indicative of a physiological measure of the one or more occupant; and
one or more controllers in electronic communication with the one or more cameras and the one or more biometric sensors, wherein the one or more controllers execute instructions to:
determine one or more physiological measures of an occupant based on the image data collected by the one or more cameras and one or more parameters indicative of the one or more physiological measures of each occupant collected by the one or more biometric sensors;
compare a physiological measure of the occupant with a baseline value, wherein the baseline value for each physiological measurement is indicative of the occupant functioning normally;
in response to determining the physiological measure falls outside an allowable range of the baseline value, compare the physiological measure with a major event threshold value; and
in response to determining the physiological measure exceeds the major event threshold value, generate a notification directed to a driver of the vehicle and determine the occupant requires immediate action.

20. The occupant monitoring system of claim 19, wherein the one or more controllers execute instructions to:
in response to determining the physiological measure does not exceed the major event threshold value, compare the physiological measure to a minor event threshold value;
determine the physiological measure exceeds the minor event threshold value; and
in response to determining the physiological measure exceeds the minor event threshold value, determine the occupant is experiencing a physical issue that requires monitoring by the driver of the vehicle.
